(19) United States
(12) Patent Application Publication
(10) Pub. No.: US 2005/0137437 A1
(43) Pub. Date: Jun. 23, 2005

(54) Continuous preparation of 4,4'-diisopropylbiphenyl

(75) Inventors: Grigorii Lev Soloveichik, Latham, NY (US); Olga Alexandrovna Ponomareva, Moscow (RU); Andrei Sergeevich Kuznetsov, Moscow (RU); Andrei Valentinovich Smirnov, Moscow (RU); Elena Evgenievna Knyazeva, Moscow (RU); Irina Fedorovna Moskovskaya, Moscow (RU); Irina Igorevna Ivanova, Moscow (RU)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/741,323

(22) Filed: Dec. 18, 2003

(51) Int. Cl. C07C 2/68

(52) U.S. Cl. 585/467

(57) ABSTRACT

A continuous flow process has been discovered for the highly selective isopropylation of biphenyl to 4,4′-diisopropylbiphenyl. Thus biphenyl and propene in decalin are passed through a solid zeolite catalyst bed contained in a flow reactor at moderate temperature (220° C.) and pressure (10-30 atm) together with a continuous stream of nitrogen. Surprisingly, catalyst performance is improved by the continuous introduction of the nitrogen into the reactor along with the reactants and solvent. Thus, improved performance as measured by higher percent conversion to products, higher yields of diisopropylated products and lower yields of undesired triisopropylated products are observed when a diluent gas such as nitrogen is employed. The alkylation process is selective for 4,4′-diisopropylbiphenyl using a zeolite catalyst, preferably a dealuminated mordenite in which the molar ratio of SiO₂ to Al₂O₃ moieties is in a range between about 10 to 1 and about 500 to 1.

### BACKGROUND OF THE INVENTION

This invention relates to a method for the continuous preparation of 4,4′-diisopropylbiphenyl using solid acidic catalysts.

Aromatic hydrocarbons having alkyl substituents are widely used in a variety of fields and those having a substituent at a para-position are especially important. For example, they are used as starting materials for polymers, as intermediates for dyes, drugs and agricultural chemicals, or as starting materials for liquid crystalline polymers. The present invention relates to a process for producing with good selectivity a compound having an alkyl groups at a para-position of biphenyl. 4,4′-diisopropylbiphenyl can be readily converted to 4,4′-biphenyl dicarboxylic acid or 4,4′-dihydroxybiphenyl (4,4′-biphenol) by oxidation of side hydrocarbon chains under appropriate conditions.

Known continuous processes for preparation of 4,4′-diisopropylbiphenyl by alkylation of biphenyl suffer from non-selectivity or give low yields of the target product. Attempts to prepare 4,4′-diisopropylbiphenyl in a continuous process at atmospheric pressure were limited by poor conversion and low selectivity (G. Kamalakar et al., Indian J. Chem. Technol. Vol. 6, No. 2, pp 71-74, 1999; D. Vergani et al., Applied Catalysis 163, pp 71-81, 1997). Batch type processes for the preparation of 4,4′-diisopropylbiphenyl using dealuminated zeolite catalysts demonstrate better selectivity than the known continuous processes but suffer from low productivity.

Batch methods for producing a dialkylbiphenyl such as 4,4′-diisopropylbiphenyl include (i) reacting biphenyl with an olefin or an alkyl halide in the presence of a Friedel-Crafts catalyst such as aluminum chloride (See D. B. Priddy, “Alkylation of Biphenyl Under Mild Friedel Crafts Conditions”, I & EC Product Research and Development, Vol. 8, No. 3, pp 239-241, September 1969), and (ii) reacting biphenyl with olefin or an alcohol in the presence of a solid acid catalyst such as a silica-alumina zeolite (G. S. Lee et al., Catalysis Letters, 2, pp 243-248, 1989), or other catalysts (G. Kamalakar et al., Indian J. Chem. Technol., Vol. 6, No. 2, pp 71-74, 1999).

Further, U.S. Pat. No. 5,015,797 and a published report (Catalysis Letters, 50, p 149, 1998) illustrate the preparation of 4,4′-diisopropylbiphenyl with relatively high selectivity using a dealuminated mordenite zeolite catalyst by reacting biphenyl with propene at elevated pressures. Another batch process for the preparation of 4,4′-diisopropylbiphenyl carried out in an autoclave at high temperature is presented in Catalysis Today 31, 3, (1996). Batch processes for the preparation of 4,4′-diisopropylbiphenyl are inherently limited and hence undesirable because they suffer low productivity, difficult catalyst regeneration, and like problems.

It would be highly desirable to discover a method for the preparation of 4,4′-diisopropylbiphenyl in a continuous process which did not suffer from the disadvantages of the earlier processes. The present invention overcomes these and other limitations and provides an efficient method for the continuous preparation of 4,4′-diisopropylbiphenyl.

### BRIEF SUMMARY OF THE INVENTION

The present invention provides a method for the continuous preparation of 4,4′-diisopropylbiphenyl, said method comprising:
(a) continuously contacting in a flow reactor (i) biphenyl, (ii) at least one inert solvent, (iii) propene, and (iv) an inert diluent gas, said flow reactor containing at least one solid acidic catalyst, said contacting being conducted at a pressure greater than about 1 atmosphere and at a temperature greater than 180° C.; and
(b) continuously recovering an effluent steam comprising product 4,4′-diisopropylbiphenyl, inert solvent, and inert diluent gas.

### DETAILED DESCRIPTION OF THE INVENTION

The present invention may be understood more readily by reference to the following detailed description of preferred embodiments of the invention and the examples included therein. In the following specification and the claims which follow, reference will be made to a number of terms which shall be defined to have the following meanings:

The singular forms “a”, “an” and “the” include plural referents unless the context clearly dictates otherwise.

“Optional” or “optionally” means that the subsequently described event or circumstance may or may not occur, and that the description includes instances where the event occurs and instances where it does not.

As noted the present invention relates to the continuous preparation of 4,4′-diisopropylbiphenyl I, 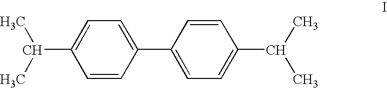
a valuable intermediate which may be used in the preparation of 4,4′-biphenol II.

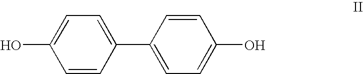

The method according to the present invention is said to be “continuous”, meaning that reactants biphenyl and propene, and inert components such as solvent and inert gas are continuously introduced into a flow reactor and a product stream is continuously removed from said flow reactor. Flow reactors useful according to the method of the present invention include tube reactors, trickle column reactors, continuous stirred tank reactors, fluidized bed reactors, and the like.

The flow reactor used according to the method of the present invention contains at least one solid acidic catalyst disposed within it. The solid acidic catalyst may be configured as a single fixed bed within the flow reactor, or may be configured as a series of multiple fixed beds arrayed along the length of the flow reactor, said multiple fixed beds being separated from one another by unused volume within the flow reactor or by an inert space-filling substance such as glass beads. Additionally, the solid acidic catalyst may have dispersed within it inert space filling substances, such as glass, inert ceramic materials, inert minerals, inert metals and the like. As mentioned, the solid acidic catalyst may also be configured as a fluidized bed, a mechanically agitated bed and the like. Additionally, the solid acidic catalyst may be configured in a “doughnut shape” wherein the catalyst is distributed along the walls of the reactor while the center of the flow reactor remains open. Reactants may be introduced into the flow reactor at a single point along the flow reactor or at multiple points along the flow reactor. Reactants biphenyl and propene may be introduced in such a manner such that they flow in opposite directions along a tube reactor, for example. Reactants and inert gas may be introduced into the flow reactor such that reactants and inert gas travel in opposite directions, for example a vertically mounted tube reactor containing a fixed or fluidized bed of the solid acidic catalyst. The tube reactor of the foregoing example has feed inlets for propene, and a solution of biphenyl in an inert solvent at the top of the reactor and an inlet for the inert gas at the bottom of the vertically mounted tube reactor. The vertically mounted tube reactor of this example is further equipped with outlets at both the top and bottom of the reactor to accommodate the outflow of products and inert gas respectively.

Regardless of the exact type of flow reactor employed or the configuration of the solid acidic catalyst within it, what is essential for the operation of the present invention is the simultaneous presence in the flow reactor of the solid acidic catalyst, the reactants (biphenyl and propene), and the inert components (solvent and inert gas), under conditions which promote product 4,4′-diisopropylbiphenyl formation. This simultaneous presence requirement is described in terms of “continuously contacting” the solid acidic catalyst with biphenyl, at least one inert solvent, propene, and an inert diluent gas.

Although not wishing to be bound by any particular theory of operation, it is believed that the greater efficiency of 4,4′-diisopropylbiphenyl production observed when an inert diluent gas is employed according to the method of the present invention is related to an enhanced removal and recovery of the product 4,4′-diisopropylbiphenyl. As a result of its enhanced removal, the product 4,4′-diisopropylbiphenyl is believed to suffer less “overalkylation” to undesired triisopropylated products. It may be that reaction systems comprised by the method of the present invention is sufficiently complex such that no simple chemical phenomenon-based theory adequately explains the enhanced performance observed. Typically, the solid acidic catalyst used according to the method of the present invention is an acidic zeolite, for example zeolites of type A, X, Y, USY, and ZSM-5 which are available commercially and contain varying amounts of SiO₂ and Al₂O₃ structural units. Mordenite type zeolites CBV 10A, CVB 21A, and CVB 90A have been found to be suitable acidic catalysts for the practice of the present invention. Mordenite type zeolites CBV 10A, CVB 21A, and CVB 90A are available from the Zeolyst Corporation Valley Forge, Pa. (USA) (www.zeolyst.com).

As mentioned, the acidic catalyst used according to the method of the present invention is typically an acidic zeolite comprising SiO₂ and Al₂O₃ structural units, said SiO₂ and Al₂O₃ structural units being present in an amount such that the molar ratio of SiO₂ to Al₂O₃ structural units is in a range from about 10 SiO₂ structural units for each Al₂O₃ structural unit to about 500 SiO₂ structural units for each Al₂O₃ structural units. Expressed in terms of silicon and aluminum content the molar ratio of “Si” to “Al” present in the zeolite is in a range between about 5:1 and about 250:1. Molar ratios of silicon to aluminium greater than about 10 to 1 are generally preferred.

Typically, the reactant biphenyl is introduced into the flow reactor as a solution in an inert solvent. The choice of solvent is not particularly critical as long as the solvent is chemically inert. Typically, the inert solvent used according to the method of the present invention is an inert hydrocarbon solvent having a boiling point in a range between about 80° C. and about 320° C., preferably between about 180° C. and about 320° C. Inert solvents which may be used according to the method of the present invention include saturated hydrocarbons such as decalin, decane, dodecane, tetradecane, hexadecane, and the like.

Typically, the solution of biphenyl in an inert solvent is continuously introduced into the flow reactor at a rate such that the weight hourly space velocity (WHSV) of biphenyl with respect to solid acidic catalyst is between about 0.025 hr⁻¹ and about 10 hr¹. In certain preferred embodiments the weight hourly space velocity of biphenyl with respect to the solid acidic catalyst is between about 0.1 hr⁻¹ and about 2.5 hr−¹. In instances in which the biphenyl is introduced independently from the inert solvent the weight hourly space velocity of biphenyl with respect to the solid acid catalyst falls within the same range of rates of introduction, namely between about 0.025 hr⁻¹ and about 10 hr¹.

Propene is typically introduced into the flow reactor as a gas. The propene may be introduced in pure form or as a mixture with an inert gas, for example a mixture of propene with argon. The propene may be introduced through a single or a plurality of feed inlets on the flow reactor. Typically, the feed inlet through which the propene is introduced is distinct from that used to introduce biphenyl. The propene may be introduced at any point along the flow reactor but is typically introduced at a position located above the acidic catalyst bed or below the acidic catalyst in for example a vertically mounted tubular flow reactor. In one embodiment, the acidic catalyst is disposed within a vertically mounted tube reactor, said tube reactor having a top and a bottom. In this embodiment, propene, a solution of biphenyl in an inert solvent, and an inert gas are introduced from the top of said flow reactor. An effluent stream consisting of products, starting materials and inert gas exits the bottom of the reactor.

Typically, propene is introduced into the flow reactor at a rate corresponding to between about 0.1 and about 10 moles of propene per mole of biphenyl introduced. For example, an embodiment of the present invention wherein the rate of introduction of biphenyl corresponds to 1 mole of biphenyl being introduced into the flow reactor per hour and the rate of propene introduction corresponds to 10 moles of propene being introduced into the flow reactor per hour, the rate of propene introduction is said to correspond to “10 moles of propene per mole of biphenyl” introduced and propene is said to be “present” in the flow reactor in an amount corresponding to 10 moles of propene per mole of biphenyl. In one embodiment of the present invention propene is present in the flow reactor in an amount corresponding to between about 2 and about 5 moles of propene per mole of biphenyl.

The diluent gas may be any gas which is inert under the reaction conditions and which does not interfere with the production of 4,4′-diisopropylbiphenyl. For example the inert gas used according to the method of the present invention may be nitrogen, helium, argon, carbon dioxide, or a mixture of two or more of these gases. Typically, nitrogen is preferred. The amount of inert gas employed according to the method of the present invention has been found to impact the yield of 4,4′-diisopropylbiphenyl. It has been found that increasing the space velocity of the inert gas relative to the biphenyl and propene reactants enhances the yield of 4,4′-diisopropylbiphenyl. In addition it has been found that increasing the space velocity of the inert gas relative to the biphenyl and propene reactants enhances the “selectivity” for 4,4′-diisopropylbiphenyl. “Selectivity” for 4,4′-diisopropylbiphenyl is defined as the number of moles of 4,4′-diisopropylbiphenyl contained in the reactor effluent expressed as a percentage of the total number of moles of isopropylated products present in the reactor effluent. Typically, the amount of inert gas employed is in a range between about 1 and about 100 moles of inert gas per mole of biphenyl introduced into the flow reactor. In one embodiment of the present invention the amount of inert gas introduced into the flow reactor is in a range between about 5 and about 75 moles of inert gas per mole of biphenyl introduced into the flow reactor. In an alternate embodiment the amount of inert gas introduced into the flow reactor is in a range between about 10 and about 40 moles of inert gas per mole of biphenyl introduced into the flow reactor.

The method of the present invention can be carried out at any pressure but is preferably carried out at supraatmospheric pressure. In one embodiment the flow reactor is operated at a pressure in a range between about 1 and about 100 atmospheres. In an alternate embodiment the flow reactor is operated at a pressure in a range between about 1 and about 75 atmospheres. In yet another embodiment the flow reactor is operated at a pressure in a range between about 2 and about 40 atmospheres. The pressure in the flow reactor may be controlled by a variety of engineering means, such as a backpressure regulator located downstream of the point at which the reactor effluent emerges from the flow reactor.

Typically, the reactor is operated at a temperature sufficient to effect the conversion of a significant fraction of the biphenyl starting material to product 4,4′-diisopropylbiphenyl. By a “significant amount” it is meant that more than about 1 percent, preferably more than about 5 percent, and still more preferably more than about 10 percent of the biphenyl introduced into the reactor is converted to 4,4′-diisopropylbiphenyl (DIPBP) or the intermediate 4-isopropylbiphenyl (IPBP). Typically, the flow reactor is operated at a temperature of between about 180° C. and about 320° C. In one embodiment, the flow reactor is operated at a temperature in a range between about 200° C. and about 250° C.

In instances in which the biphenyl is introduced into the flow reactor as a solution in an inert solvent it is useful to gauge the feed rate of this solution in terms of “catalyst bed volumes per hour”. The rate of solution introduction expressed in bed volumes per hour corresponds to the volume of solution introduced into the flow reactor over the course of one hour divided by the volume of the solid catalyst disposed within the flow reactor. Alternatively, when the inert solvent and biphenyl are introduced separately it is still frequently useful to describe the rate of biphenyl and solvent introduction in terms of catalyst bed volumes per hour. In this instance the volumes of solvent and biphenyl separately introduced over the course of an hour are combined and divided by the volume of the solid catalyst disposed within the reactor. Typically the rate of biphenyl and inert solvent introduction into the flow reactor is between about 0.1 and about 5 catalyst bed volumes per hour. In one embodiment biphenyl is introduced into the flow reactor as a solution in at least one inert solvent at a rate corresponding to between about 1 and about 3 bed volumes per hour.

One embodiment the present invention provides a method for the preparation of 4,4′-diisopropylbiphenyl, said method comprising:

(a) continuously introducing into a flow reactor containing a solid bed of an acidic zeolite catalyst at a temperature in a range between about 180° C. and about 320° C. and a pressure between about 2 and about 50 atmospheres, (i) a solution comprising biphenyl and at least one inert hydrocarbon solvent, (ii) propene, and (iii) an inert diluent gas, said solvent having a boiling point in a range between about 180° C. and about 320° C., said solution being introduced into said flow reactor at a feed rate corresponding to about 0.1 to about 5 catalyst bed volumes per hour, said propene being introduced at a feed rate corresponding to between about 1 and about 10 moles of propene per mole of biphenyl; and

(b) continuously removing from the flow reactor an effluent steam comprising product 4,4′-diisopropylbiphenyl, inert hydrocarbon solvent, and inert diluent gas.

An alternate embodiment of the present invention provides a method for the preparation of 4,4′-diisopropylbiphenyl, said method comprising:

(a) continuously introducing into a flow reactor containing a solid bed of an acidic zeolite catalyst at a temperature in a range between about 200° C. and about 250° C. and a pressure between about 10 and about 30 atmospheres, (i) a solution comprising biphenyl and decalin, (ii) propene, and (iii) nitrogen gas, said solution having a concentration of biphenyl in a range between about 0.1 and 1.0 moles of biphenyl per liter of solution, said solution being introduced into said flow reactor at a feed rate corresponding to between about 0.1 and about 5 catalyst bed volumes per hour, said propene being introduced at a feed rate corresponding to between about 1 and about 10 moles of propene per mole of biphenyl; and

(b) continuously removing from the flow reactor an effluent steam comprising product 4,4′diisopropylbiphenyl, decalin, and nitrogen gas.

### EXAMPLES

The following examples are set forth to provide those of ordinary skill in the art with a detailed description of how the methods claimed herein are evaluated, and are not intended to limit the scope of what the inventors regard as their invention. Unless indicated otherwise, parts are by weight, temperature is in ° C. The term “weight hourly space velocity” (WHSV) refers to the weight in grams per hour of a component introduced into the reactor per gram of the solid zeolite catalyst contained within the reactor.

### GENERAL PROCEDURE (FOR EXAMPLES AND COMPARATIVE EXAMPLES)

A stainless steel fixed-bed type reactor having two inlets at the top of the reactor and a single outlet for effluent at the bottom of the reactor was charged with 0.7 grams of granulated CBV 10A (mordenite-type zeolite produced by ZEOLYST having a molar ratio of SiO₂ to Al₂O₃ of about 13) which had been calcined in a flow of dry air at 550° C. for 6 hours prior to its use. The reactor was operated in the vertical position. Reagents and nitrogen were introduced through the two inlets at the top of the reactor and the reactor effluent emerged through an outlet at the bottom of the reactor. The granulated CBV 10A catalyst had a grain size of from about 0.25 to about 0.5 mm. A 0.8 molar (0.8M) solution of biphenyl in decalin was prepared. Propene was added to the 0.8 M biphenyl in decalin solution in the amounts indicated in Table 1 and the resulting solution was then fed into the reactor with a syringe metering pump at about 2 to about 3 grams of the solution per hour through one of the two reactor inlets. In the Examples, nitrogen gas was introduced through the second reactor inlet. The reactor temperature was about 220° C. as measured by a thermocouple in contact with the solid catalyst located within the reactor, the reactor pressure was about 30 atmospheres, the molar ratio of biphenyl to propylene was either about 1:2 or about 1:5, and the weight hourly space velocity (WHSV) with respect to biphenyl was about 0.5 hr⁻¹. Each experiment was run continuously for a period of from about 2 to about 20 hours. The reactor effluent was analyzed by gas chromatography (GC) to determine the distribution of products and starting materials. The data for the Examples and Comparative Examples are presented in Table 1 and represent reaction behavior under steady state conditions. Steady state conditions refer to the period of stable operation of the reactor wherein the percent conversion of reactants to products was neither rising nor declining and was deemed to begin at a point in time following the initial reactor start-up. The end of the steady state period was marked by a decline in percent conversion of starting materials to products. Comparative Examples are marked with an asterisk (*). It should be noted that in the various Examples and Comparative Examples the pressure within the reactor was controlled by a back pressure regulator at the reactor outlet. The pressure in the system was maintained at a given level by adjusting the back pressure regulator to achieve the desired reactor pressure. In this way substantial amounts of nitrogen could be introduced simultaneously with reactants and solvents without resulting in an increase in the overall pressure.

### Example 1* (Comparative)

The reaction was carried out as described in the GENERAL PROCEDURE described above. No nitrogen diluent was employed.

### Example 2

The procedure used was the same as in the GENERAL PROCEDURE described above. Nitrogen gas was used as a diluent and was delivered using a mass flow controller at a rate of about 7.3 milliliters per minute. The relative molar amounts of biphenyl (BP), propene (Pr) and nitrogen (N₂) being introduced were 1 mole BP per 2 moles Pr per 10 moles N₂. This is expressed in shorthand as “BP:Pr:N₂=1:2:10 (mole)”. After passing through the reactor and back pressure regulator, the gas and liquid phases of the effluent were separated at atmospheric pressure. Product analysis was carried out by GC. The results are shown in Table 1.

### Example 3

The reaction was carried out as described in Example 2 except that the ratio BP:Pr:N2 was 1:2:20 (mole). The results are shown in Table 1.

### Example 4* (Comparative)

The reaction was carried out as described in Example 1* except that the molar ratio of biphenyl to propene was 1:5 (“BP:Pr”=1:5 (mole)). No nitrogen diluent was employed. The results are shown in Table 1.

### Examples 5-7

The reaction was carried out as described in Example 2 except that the ratio BP:Pr:N2 was 1:5:10 (mole) to 1:5:40 (mole). Thus in Examples 5-7 the ratio of BP to Pr was 1:5 while the ratio of BP to N₂ varied from 1:10 (mole) (Example 5) to 1:40 (mole) (Example 7). The results are shown in Table 1.

### Example 8* (Comparative)

The reaction was carried out as in Example 4 except that the reaction pressure was 10 atmospheres (atm). The results are shown in Table 1.

### Examples 9-11

The reaction was carried out as in Example 8 except that nitrogen was employed as a diluent. The ratio BP:N2 (mole) varied from 1:10 to 1:40. The results are shown in Table 1.

As noted, the catalyst employed in each Example and Comparative Example (*) was the CBV 10A acidic zeolite described earlier. In Table 1 the reaction pressure is given in atmospheres in the column headed “P(atm). The heading “WHSV” refers to the weight hourly space velocity with respect to the biphenyl (BP) introduced. The column headed “BP/Pr/N₂” provides values of the molar ratios of biphenyl (BP) to propene (Pr) to nitrogen (N₂). Comparative Examples (*) did not employ nitrogen and thus indicate only the molar ratio of biphenyl to propene (BP/Pr). “% Conv.” Refers to the extent to which biphenyl is converted to products, the products being isopropylbiphenyl, diisopropylbiphenyl, and triisopropylbiphenyl (“IPBP”, “DIPBP” and “TIPBP” respectively). Column headings “% IPBP”, “% DIPBP” and “% TIPBP” refer to relative amounts of isopropylbiphenyl, diisopropylbiphenyl, and triisopropylbiphenyl present in the reactor effluent as determined by GC. “Selectivity” refers to the amount of 4,4′-diisopropylbiphenyl present in the reactor effluent relative to other isomers of diisopropylbiphenyl (DIPBP). “% Yield” refers to the percent yield of 4,4′-diisopropylbiphenyl. For example, in Comparative Example 1* the percent yield of 4,4′-diisopropylbiphenyl is give as 14.5 percent (50.5 percent conversion×0.379% DIPBP×0.76 Selectivity=14.5%).

The data in Table 1 illustrate the present invention and demonstrate the surprising effect of use of a diluent gas on the yield of 4,4′-diisopropylbiphenyl. Thus, in Examples 2 and 3 the yield of 4,4′-diisopropylbiphenyl is dramatically increased. This effect is observed at higher propene to biphenyl ratios as well (See Comparative Example 4* and Examples 5-7 in which the molar ratio of propene to biphenyl is 5 to 1.). Interestingly, the amount of product lost to “overalkylation”, i.e. the conversion of DIPBP to TIPBP, is reduced through the use of nitrogen as the diluent gas.

TABLE 1
CONTINUOUS REACTION OF BIPHENYL WITH PROPENE OVER A
SOLID ACIDIC ZEOLYTE CATALYST

| Example | P(atm) | WHSV | BP/Pr/N₂ | % Conv. | % IPBP | % DIPBP | % TIPB | Selectivity | % Yield |
|---|---|---|---|---|---|---|---|---|---|
| 1* | 30 | 0.5 | 1:2 | 50.3 | 47.4 | 37.9 | 3.7 | 76.1 | 14.5 |
| 2 | 30 | 0.5 | 1:2:10 | 62.2 | 41.2 | 48.7 | 2.3 | 77.4 | 23.4 |
| 3 | 30 | 0.5 | 1:2:20 | 64.7 | 39.3 | 51.2 | 2.0 | 78.2 | 25.9 |
| 4* | 30 | 0.5 | 1:5 | 67.5 | 32.6 | 50.3 | 8.6 | 70.6 | 24.0 |
| 5 | 30 | 0.5 | 1:5:10 | 82.3 | 24.2 | 61.9 | 6.0 | 71.1 | 36.2 |
| 6 | 30 | 0.5 | 1:5:20 | 84.8 | 22.4 | 64.4 | 6.4 | 70.3 | 38.4 |
| 7 | 30 | 0.5 | 1:5:40 | 85.8 | 21.4 | 66.8 | 5.2 | 69.9 | 40.1 |
| 8* | 10 | 0.5 | 1:5 | 76.5 | 28.5 | 58.1 | 6.5 | 73.4 | 32.6 |
| 9 | 10 | 0.5 | 1:5:10 | 86.8 | 22.2 | 66.6 | 4.7 | 73.4 | 42.5 |
| 10 | 10 | 0.5 | 1:5:20 | 87.6 | 20.9 | 68.8 | 4.6 | 71.4 | 43.0 |
| 11 | 10 | 0.5 | 1:5:40 | 82.6 | 14.2 | 77.0 | 4.0 | 70.0 | 44.5 |

*Indicates a Comparative Example

The invention has been described in detail with particular reference to preferred embodiments thereof, but it will be understood by those skilled in the art that variations and modifications can be effected within the spirit and scope of the invention.

1. A method for the preparation of 4,4′-diisopropylbiphenyl, said method comprising:
(a) continuously contacting in a flow reactor (i) biphenyl, (ii) at least one inert solvent, (iii) propene, and (iv) an inert diluent gas, said flow reactor containing at least one solid acidic catalyst, said contacting being conducted at a pressure greater than about 1 atmosphere and at a temperature greater than 180° C.; and
(b) continuously recovering an effluent steam comprising product 4,4′-diisopropylbiphenyl, inert solvent, and inert diluent gas.

2. A method according to claim 1 wherein said solid acid catalyst is selected from the group consisting of acidic zeolites of type A, X, Y, USY and ZSM-5.

3. A method according to claim 1 wherein said acidic zeolite is a mordenite acidic zeolite.

4. A method according to claim 3 wherein said acidic zeolite comprises SiO₂ and Al₂O₃ structural units, said SiO₂ and Al₂O₃ structural units being present in a molar ratio in a range from about 10 to 1 to about 500 to 1 moles of SiO₂ structural units to moles of Al₂O₃ structural units.

5. A method according to claim 1 wherein the biphenyl is continuously contacted with the solid acidic catalyst at a weight hourly space velocity (WHSV) of between about 0.025 hr⁻¹ and about 10 hr⁻¹.

6. A method according to claim 5 wherein said weight hourly space velocity (WHSV) is in a range between about 0.1 and about 2.5 hr⁻¹.

7. A method according to claim 1 wherein said inert solvent has a boiling point in a range between about 180° C. and about 320° C.

8. A method according to claim 7 wherein said solvent is a saturated hydrocarbon.

9. A method according to claim 1 wherein said propene is present in an amount corresponding to between about 0.1 mole and about 10 moles of propene per mole of biphenyl.

10. A method according to claim 9 wherein said propene is present in an amount corresponding to between about 2 and about 5 moles of propene per mole of biphenyl.

11. A method according to claim 1 wherein said diluent gas is selected from the group consisting of nitrogen, helium, argon, and carbon dioxide.

12. A method according to claim 1 wherein said diluent gas is nitrogen.

13. A method according to claim 1 wherein said contacting is conducted at a pressure in a range between about 1 atmosphere and about 100 atmospheres.

14. A method according to claim 13 wherein said contacting is carried out at a pressure in a range between about 2 atmospheres and about 40 atmospheres.

15. A method according to claim 1 wherein said contacting is carried out at a temperature in a range between about 180° C. and about 320° C.

16. A method according to claim 15 wherein said contacting is carried out at a temperature in a range between about 200° C. and about 250° C.

17. A method according to claim 1 wherein said biphenyl and said inert solvent are continuously contacted with said acidic catalyst at a feed rate corresponding to between about 0.1 and about 5 catalyst bed volumes per hour.

18. A method according to claim 17 wherein said feed rate corresponds to between about 1 and about 3 catalyst bed volumes per hour.

19. A method for the continuous preparation of 4,4′-diisopropyl biphenyl, said method comprising:
(a) continuously introducing into a flow reactor containing a solid bed of an acidic zeolite catalyst at a temperature in a range between about 180° C. and about 320° C. and a pressure between about 2 and about 50 atmospheres, (i) a solution comprising biphenyl and at least one inert hydrocarbon solvent, (ii) propene, and (iii) an inert diluent gas, said solvent having a boiling point in a range between about 180° C. and about 320° C., said solution being introduced into said flow reactor at a feed rate corresponding to about 0.1 to about 5 catalyst bed volumes per hour, said propene being introduced at a feed rate corresponding to between about 1 and about 10 moles of propene per mole of biphenyl; and
(b) continuously removing from the flow reactor an effluent steam comprising product 4,4′-diisopropylbiphenyl, inert hydrocarbon solvent, and inert diluent gas.

20. A method according to claim 19 wherein said acidic zeolite catalyst comprises SiO₂ and Al₂O₃ structural units, said SiO₂ and Al₂O₃ structural units being present in a molar ratio in a range from about 20 to 1 to about 500 to about 1 moles of SiO₂ structural units to moles of Al₂O₃ structural units.

21. A method according to claim 20 wherein the biphenyl is continuously contacted with the acidic zeolite catalyst at a weight hourly space velocity (WHSV) of between about 0.025 to about 10 hr¹.

22. A method for the continuous preparation of 4,4′-diisopropylbiphenyl, said method comprising:
(a) continuously introducing into a flow reactor containing a solid bed of an acidic zeolite catalyst at a temperature in a range between about 200° C. and about 250° C. and a pressure between about 10 and about 30 atmospheres, (i) a solution comprising biphenyl and decalin, (ii) propene, and (iii) nitrogen gas, said solution having a concentration of biphenyl in a range between about 0.1 and 1.0 moles of biphenyl per liter of solution, said solution being introduced into said flow reactor at a feed rate corresponding to between about 0.1 and about 5 catalyst bed volumes per hour, said propene being introduced at a feed rate corresponding to between about 1 and about 10 moles of propene per mole of biphenyl; and
(b) continuously removing from the flow reactor an effluent steam comprising product 4,4′-diisopropylbiphenyl, decalin, and nitrogen gas.
